(19) United States
(12) Patent Application Publication
(10) Pub. No.: US 2022/0168314 A1
(43) Pub. Date: Jun. 2, 2022

(54) MEDICAMENT FOR PROPHYLAXIS OR TREATMENT OF PULMONARY FIBROSIS

(71) Applicant: MITSUBISHI TANABE PHARMA CORPORATION, Chuo-ku (JP)

(72) Inventors: Anthony AZZARA, Pennington, NJ (US); Kohei KIKKAWA, Chuo-ku (JP)

(73) Assignee: MITSUBISHI TANABE PHARMA CORPORATION, Chuo-ku (JP)

(21) Appl. No.: 17/425,810

(22) PCT Filed: Feb. 4, 2020
(86) PCT No.: PCT/US2020/016491

§ 371 Date: Jul. 26, 2021

Related U.S. Application Data
(60) Provisional application No. 62/801,172, filed on Feb. 5, 2019.

(51) Int. Cl. A61K 31/5375; A61P 11/00

(52) U.S. Cl. CPC A61K 31/5375; A61P 11/00

(57) ABSTRACT

A medicament for prophylaxis or treatment of pulmonary fibrosis containing as an active ingredient a compound represented by the following formula (I) or a pharmaceutically acceptable salt thereof.

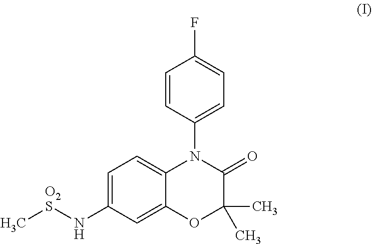

### CROSS-REFERENCE TO RELATED APPLICATIONS

The present application is based upon and claims the benefit of priority from U.S. Provisional Application No. 62/801,172, filed Feb. 5, 2019, the entire contents of which are incorporated herein by reference.

### BACKGROUND OF THE INVENTION

### Field of the Invention

The present invention relates to a medicament for prophylaxis or treatment of pulmonary fibrosis and a method for prophylaxis or treatment of pulmonary fibrosis, and is useful in the field of medicine.

### Description of Background Art

In International Publication No. 2007/089034, a 1,4-benzoxazine compound is described, and it is described that a compound (I) is used as an MR antagonist. Further, in International Publication No. 2018/062134, it is described that the compound (I) is used for treatment of a non-alcoholic fatty liver disease (NAFLD), non-alcoholic steatohepatitis (NASH), and the like. The entire contents of this publication are incorporated herein by reference.

### SUMMARY OF THE INVENTION

One aspect of the present invention is a medicament for prophylaxis or treatment of pulmonary fibrosis containing as an active ingredient a compound (hereinafter, may be referred to as a compound (I)) represented by the following formula (I)

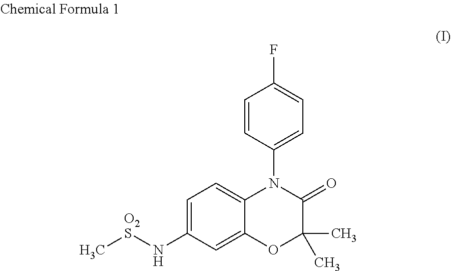

or a pharmaceutically acceptable salt thereof.

The medicament may be for the prophylaxis or treatment of pulmonary fibrosis, in which the pulmonary fibrosis is an interstitial lung disease with fibrosis.

The medicament may be for the prophylaxis or treatment of pulmonary fibrosis, in which the interstitial lung disease with fibrosis is a disease selected from a group consisting of idiopathic pulmonary fibrosis, disease induced lung fibrosis and other factor induced lung fibrosis.

Another aspect of the present invention is a pharmaceutical composition for prophylaxis or treatment of pulmonary fibrosis containing as an active ingredient a compound represented by the following formula (I)

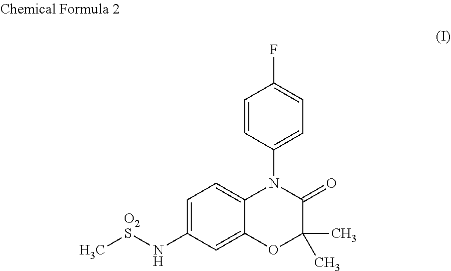

or a pharmaceutically acceptable salt thereof, together with a pharmaceutically acceptable carrier.

The pharmaceutical composition may be for the prophylaxis or treatment of pulmonary fibrosis, in which the pulmonary fibrosis is an interstitial lung disease with fibrosis.

The pharmaceutical composition may be for the prophylaxis or treatment of pulmonary fibrosis, in which the interstitial lung disease with fibrosis is a disease selected from a group consisting of idiopathic pulmonary fibrosis, disease induced lung fibrosis and other factor induced lung fibrosis.

Yet another aspect of the present invention is a method for prophylaxis or treatment of pulmonary fibrosis including administering a prophylactically or therapeutically effective amount of a compound represented by the following formula (I)

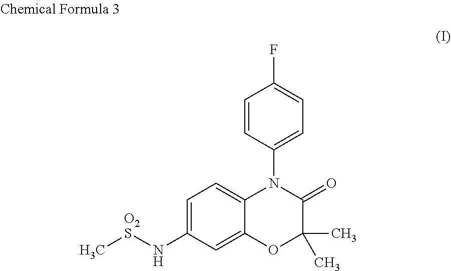

or a pharmaceutically acceptable salt thereof to a patient in need thereof.

The method may be for the prophylaxis or treatment of pulmonary fibrosis, in which the pulmonary fibrosis is an interstitial lung disease with fibrosis.

The method may be for the prophylaxis or treatment of pulmonary fibrosis, in which the interstitial lung disease with fibrosis is a disease selected from a group consisting of idiopathic pulmonary fibrosis, disease induced lung fibrosis and other factor induced lung fibrosis.

Still another aspect of the present invention is a compound represented by the following formula (I)

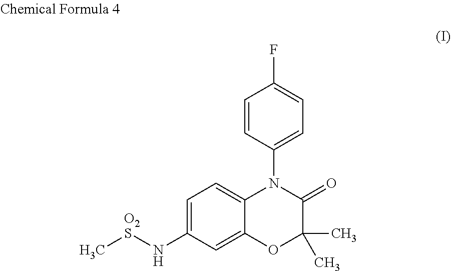

or a pharmaceutically acceptable salt thereof for prophylaxis or treatment of pulmonary fibrosis.

The compound or a pharmaceutically acceptable salt thereof may be for the prophylaxis or treatment of pulmonary fibrosis, in which the pulmonary fibrosis is an interstitial lung disease with fibrosis.

The compound or a pharmaceutically acceptable salt thereof may be for the prophylaxis or treatment of pulmonary fibrosis, in which the interstitial lung disease with fibrosis is a disease selected from a group consisting of idiopathic pulmonary fibrosis, disease induced lung fibrosis and other factor induced lung fibrosis.

Still another aspect of the present invention is use of a compound represented by the following formula (I)

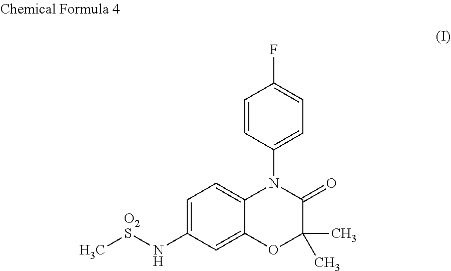

or a pharmaceutically acceptable salt thereof for manufacturing a medicament for prophylaxis or treatment of pulmonary fibrosis.

The use of the compound or the pharmaceutically acceptable salt thereof, in which the pulmonary fibrosis is an interstitial lung disease with fibrosis.

The use of the compound or the pharmaceutically acceptable salt thereof, in which the interstitial lung disease with fibrosis is a disease selected from a group consisting of idiopathic pulmonary fibrosis, disease induced lung fibrosis and other factor induced lung fibrosis.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawings, wherein:

FIG. 1 shows results of evaluation of an inspiratory capacity and elastance performed in Example 1;

FIG. 2 shows results of evaluation of lung compliance and resistance performed in Example 1;

FIG. 3 shows results of evaluation of a hydroxyproline content and a positive rate in picrosirius red staining performed in Example 1;

FIG. 4 shows results of evaluation of collagen gene expression performed in Example 1;

FIG. 5 shows results of evaluation of αSMA, fibronectin and vimentin gene expressions performed in Example 1;

FIG. 6 shows results of evaluation of CTGF, PAI-1 and Ccl2 gene expressions performed in Example 1;

FIG. 7 shows results of evaluation of TGFβ gene expression performed in Example 1;

FIG. 8 shows results of evaluation of a concentration of the compound (I) in a blood plasma performed in Example 1;

FIG. 9 shows results of evaluation of an inspiratory capacity and resistance performed in Example 2;

FIG. 10 shows results of evaluation of elastance and compliance performed in Example 2;

FIG. 11 shows results of evaluation of a hydroxyproline content and a positive rate in picrosirius red staining performed in Example 2; and

FIG. 12 shows results of evaluation of a concentration of the compound (I) in a blood plasma performed in Example 2.

### DETAILED DESCRIPTION OF THE EMBODIMENTS

Embodiments will now be described with reference to the accompanying drawings, wherein like reference numerals designate corresponding or identical elements throughout the various drawings.

In the present specification, an example of “pulmonary fibrosis” which is a target disease is an “interstitial lung disease with fibrosis.” Further, in the present specification, all “fibers” generated in a living body are expressed as “fibers.” Examples of “interstitial lung disease with fibrosis” include “idiopathic pulmonary fibrosis (IPF),” “disease induced lung fibrosis” and “other factor induced lung fibrosis.” Here, the term “idiopathic pulmonary fibrosis” refers to pulmonary fibrosis for which a cause cannot be identified. The term “disease induced lung fibrosis” refers to pulmonary fibrosis that occurs in conjunction with a disease among pulmonary fibroses. For example, the “disease induced lung fibrosis” is pulmonary fibrosis in which fibrosis has developed from hypersensitivity pneumonia (HP), a rheumatoid arthritis-associated interstitial lung disease (RA-ILD), a systemic scleroderma-associated interstitial lung disease (SSc-ILD), a polymyositis/dermatomyositis-associated interstitial lung disease (PM/DM-ILD), a Sjogren's syndrome-associated interstitial lung disease (Sjogren's ILD), a systemic lupus erythematosus-associated interstitial lung disease (SLE-ILD), a mixed connective tissue disease-associated interstitial lung disease (MCTD-ILD), a collagen disease-associated interstitial lung disease (CTD-ILD), pulmonary sarcoidosis, or the like.

The “other factor induced lung fibrosis” is, for example, pulmonary fibrosis that is a pulmonary disease caused by idiopathic nonspecific interstitial pneumonia (iNSIP), exposure to an inorganic substance, exposure to an organic substance, a drug or smoking, or the like, and in which fibrosis has developed.

In the present specification, the term “treatment” includes cure of a disease (all pathological conditions or one or more pathological conditions), improvement of the disease, and suppression of progression of severity of the disease. The term “therapeutically effective amount” refers to a dosage of the compound (I) sufficient to achieve such a purpose.

The therapeutic medicament of the present specification can also be used as a prophylactic medicament. The term “prophylaxis” includes prophylaxis of development of a disease (all pathological conditions or one or more pathological conditions) and delay of the development of the disease. The term “prophylactically effective amount” refers to a dosage of the compound (I) sufficient to achieve such a purpose.

### Compound

The compound (I) of the present invention is a compound described in International Publication No. 2007/089034 (Example 9). A person skilled in the art can produce the compound (I) using a method described in the publication or a method conforming thereto.

In practicing the present embodiment, the compound (I) can be used in a free form or in a form of a pharmaceutically acceptable salt thereof.

In the present specification, examples of such a pharmaceutically acceptable salt of the compound (I) include salts with acids such as salts with inorganic acids such as a hydrochloride, a hydrobromide, a sulfate, and a phosphate, and salts with organic acids such as an acetate, a fumarate, an oxalate, a citrate, a methanesulfonate, a benzenesulfonate, a tosylate, and a maleate; salts with bases such as alkali metal salts such as a sodium salt and a potassium salt, and alkaline earth metal salts such as a calcium salt; salts with amino acids such as a glycine salt, a lysine salt, an arginine salt, an ornithine salt, a glutamate, and an aspartate; and the like.

The compound (I) or a pharmaceutically acceptable salt thereof includes an intramolecular salt or an adduct thereof, and also includes a solvate or a hydrate thereof.

Further, existence of crystalline polymorphism in the compound (I) is known (see International Publication No. 2014/024950). Therefore, in the present invention, the compound (I) as an active ingredient can be used in any crystalline form based on such crystalline polymorphism.

### Pharmaceutical Formulation

In practicing the present invention, the compound (I) or a pharmaceutically acceptable salt thereof (hereinafter, these may be collectively referred to as “the compound of the present invention”) can be used in a standalone form or in a form of a pharmaceutical composition containing the compound of the present invention as an active ingredient together with a pharmaceutically acceptable carrier.

Examples of such a pharmaceutical composition include tablets, pills, powders, granules, capsules and emulsions.

In the present specification, as the “pharmaceutically acceptable carrier,” various carriers commonly used in the field of pharmaceutical formulation technology can be used.

As specific examples of the “pharmaceutically acceptable carrier,” for example, in solid pharmaceutical formulation, an excipient, a lubricant, a binding agent, and a disintegrating agent can be used.

In a liquid pharmaceutical formulation, a vehicle, a dissolution aid, a suspending agent, an isotonizing agent, and a buffering agent can be used.

When necessary, other necessary additives such as an antiseptic agent may be blended.

The pharmaceutical composition of the present invention varies depending on a dosage form, an administration method, a carrier, and the like, but can be produced by adding the compound of the present invention in an amount of usually 0.01-99% (w/w), and preferably 0.1-85% (w/w) with respect to a total amount of a pharmaceutical formulation. The pharmaceutical composition, depending on a form thereof, can be produced using a commonly used method in the field of pharmaceutical formulation technology. The pharmaceutical composition of the present invention may be molded into a sustained-release pharmaceutical formulation containing an active ingredient.

In the above, the compound of the present invention and the pharmaceutical composition of the present invention have been described. The compound of the present invention and the pharmaceutical composition of the present invention are expected to have the following excellent effects.

For example, when a steroid type MR antagonist such as spironolactone or eplerenone is used, there is a concern about serious side effects (for example, gynecomastia, menstrual irregularities, erectile dysfunction, and the like). However, for the compound of the present invention and the pharmaceutical composition of the present invention, there is little concern about such serious side effects, and thus, the compound of the present invention and the pharmaceutical composition of the present invention can have high safety as a medicament.

In addition, eplerenone is mainly metabolized by CYP3A4, and thus, is contraindicated for use in combination with a strong CYP3A4 inhibitor. However, in the case of the compound of the present invention, a metabolic pathway is different, and thus, the compound of the present invention is not subjected to such a restriction and can be used in combination with a wide range of other drugs. Therefore, the compound of the present invention and the pharmaceutical composition of the present invention are highly useful in clinical practice.

Further, the compound of the present invention and the pharmaceutical composition of the present invention have a feature in terms of pharmacokinetics that a constant drug level in a blood plasma can be maintained for a long period of time, and thus, can exert a sustained effect even at a low dose. Therefore, also from this point of view, the compound of the present invention and the pharmaceutical composition of the present invention can be used as a medicament having low toxicity and high safety.

### Subject to be Administered

As described above, the compound of the present invention has low toxicity and is expected to have few side effects, and also has excellent properties as a medicament. Therefore, the compound of the present invention can be safely administered to a mammal (in particular, a human).

### Route of Administration

In practicing the present invention, the compound of the present invention can be independently, or as a pharmaceutical composition, administered orally or parenterally (for example, intravenous, intramuscular, subcutaneous, intra-organ, intranasal, intradermal, ophthalmic, intracerebral, intrarectal, intravaginal and intraperitoneal administrations, and administration to a lesion).

### Dosage

A dosage of the compound of the present invention varies depending on a subject to be administered, a route of administration and an age and symptoms of a subject to be administered, but is not particularly limited. For example, when the compound of the present invention is orally administered to an adult patient of pulmonary fibrosis (having a body weight of about 40-80 kg, for example, 60 kg), a dosage thereof per day is, for example, 1-30 mg, preferably 1-25 mg, even more preferably 2.5-25 mg, and particularly preferably 7.5-25 mg. This amount can be administered at a dosing schedule of 1-3 times a day.

Use in Combination with Other Drugs

As described above, the compound of the present invention has extremely low toxicity and can be used in combination with other drugs in prophylaxis or treatment of pulmonary fibrosis, and excellent prophylactic and/or therapeutic effects by combining with other drugs can be expected. Further, it can be expected that such combination therapy reduces a dose of other drugs and reduces side effects of the other drugs.

Examples of such drugs (hereinafter, abbreviated as concomitant drugs) that can be used in combination with the compound of the present invention include steroid drugs (for example, prednisolone, methylprednisolone, and the like), immunosuppressive agents (for example, cyclophosphamide, cyclosporine, and the like), and antifibrotic agents (for example, nintedanib, pirfenidone, and the like).

In an actual combination therapy, a concomitant drug can be appropriately selected in view of a type of a disease of a patient, severity of symptoms thereof, and the like.

A dosage form of a concomitant drug of the present invention is not particularly limited, and the compound of the present invention and a concomitant drug can be combined at the time of administration. For example, a concomitant drug can be used in forms such as: (1) administration of a pharmaceutical formulation containing in combination the compound of the present invention and the concomitant drug; (2) simultaneous or separate administration of two kinds of pharmaceutical formulations obtained by separately formulating the compound of the present invention and the concomitant drug through the same route of administration; and (3) simultaneous or separate administration of two kinds of pharmaceutical formulations obtained by separately formulating the compound of the present invention and the concomitant drug through different routes of administration. A preferred form can be appropriately selected according to an actual situation in clinical practice.

A pharmaceutical formulation containing in combination the compound of the present invention and a concomitant drug can be appropriately produced by a person skilled in the art according to the above-described pharmaceutical composition containing the compound of the present invention.

A dosage of a concomitant drug can be appropriately selected based on a clinically used dose. Further, a compounding ratio of the compound of the present invention and a concomitant drug can be appropriately selected depending on the disease and symptoms of the subject to be administered, the route of administration, the type of the concomitant drug to be used, and the like. Usually, the compounding ratio can be appropriately determined according to an actual situation in clinical practice based on a general clinical dosage of the concomitant drug to be used.

### EXAMPLES

In the following, the present invention is described in more detail based on examples. However, these examples do not limit the scope of the present invention. Further, unless otherwise specified, reagents, devices and materials used in the present invention are commercially available or can be appropriately prepared by a person skilled in the art.

### Example 1: Antifibrotic Action in Bleomycin Intra Airway Administration Induced Mouse Pulmonary Fibrosis Model (1)

### (1) Test Method

C57/BL6NTAC mice (male; body weight at start of a test: 25 g) (manufactured by The Jackson Laboratory) were grouped into the following 4 groups (12 mice in each group):

Group 1: normal group
Group 2: no drug administration group (control group)
Group 3: drug (the compound (I)) administration group
Group 4: drug (eplerenone; positive control) administration group

On a day (Day 0) when the test was started, bleomycin (3.25 U/kg) was administered dropwise through airways to the mice of Groups 2-4 as test groups. Instead of bleomycin, saline was administered dropwise through airways to the mice of Group 1 as a normal group. On a seventh day (Day 7) after the start of the test, with a dosing schedule of twice a day in the morning and afternoon, oral gavage administration (twice in the morning and afternoon) of a vehicle (0.1% HCO60+0.5% CMC) was started for the mice of Groups 1 and 2; a 2 mg/ml solution of the compound (I) was prepared and oral gavage administration (in the morning) thereof at a dose of 10 mg/kg and oral gavage administration (in the afternoon) of a vehicle (0.1% HCO60+0.5% CMC) were started for the mice of Group 3; and oral gavage administration (twice in the morning and afternoon) of eplerenone at a dose of 50 mg/kg was started for the mice of Group 4. Thereafter, until a 21st day (Day 21) after the start of the test, the administrations were continued at the same dosing schedules. After the administrations were completed (Day 21), the following evaluation items were examined.

### a) Evaluation of Lung Functions

The following evaluation items were measured using a commercially available respiratory and lung function evaluation system (measurement site: around an airway). Evaluation of 4 items including: 1) inspiratory capacity (mL); 2) compliance (mL/cmH₂O); 3) elastance) (cmH₂O/mL); and 4) resistance (cmH₂O.s/mL)

The “inspiratory capacity” corresponds to an amount of air discharged from the lungs at a stage when slowly breathing out to an end of a breath has completed, and is an indicator of a volume of the lungs. As pulmonary fibrosis progresses and the lung hardens, the volume of the lungs shrinks, and the inspiratory capacity decreases.

The “compliance” indicates a change in a lung capacity due to a certain change in pressure. A large compliance means that a change in the volume of the lungs is large with respect to a unit pressure change, and indicates that the lungs are easy to stretch. As pulmonary fibrosis progresses and the lungs harden, the volume of the lungs shrinks, and the compliance decreases.

The “elastance” is a value represented by a reciprocal of the compliance, and is an indicator representing the difficulty for the lungs to stretch. As pulmonary fibrosis progresses and the lungs harden, the elastance increases.

The “resistance” means an airway resistance. The resistance is a resistance received by an air current in respiration, and a larger resistance means that it is harder for air to flow through the airway.

### b) Evaluation of Lung Pathological Tissue

### (i) Evaluation by Picrosirius Red Staining (Left Lung Lobes)

Pulmonary fibrosis is caused by that activated fibroblasts are accumulated at a fibrosis site and a large amount of type I collagen is produced. Therefore, based on a degree of collagen accumulation in a lung tissue, a degree of pulmonary fibrosis can be evaluated. Type I collagen and type III collagen in a tissue are stained by picrosirius red staining. Therefore, a situation of collagen accumulation can be image-diagnosed, and quantitative evaluation can also be performed by calculating a staining positive rate.

Specifically, the above staining was performed on a tissue section of the lungs, and an area of a stained site (fibrosis site) was measured and evaluated.

### (ii) Evaluation of Hydroxyproline Content (μg) (Lower Lung Lobes and Middle Lung Lobe)

Hydroxyproline is a major component of collagen and is substantially absent in other proteins. Therefore, by measuring a content of hydroxyproline in a tissue, an amount of collagen can be evaluated.

(iii) Evaluation of Gene Expression Fluctuation Associated with Pulmonary Fibrosis (Quantification of mRNA; Fold Increase) (Upper Lung Lobes)

According to a conventional method, mRNA was extracted from a homogenized upper lung lobe, and an extremely small amount of the mRNA was quantified using a real time PCR system.

### c) Evaluation of Blood Samples

At 2 hours (before the above evaluations) and at 18 hours from the final administration of the compound (I) (Day 21), a small amount of blood was collected from a tail vein under anesthesia, and plasma was separated with an ice-cooled centrifuge, and a concentration (nM) of the compound (I) in the plasma was measured using a conventional method.

### (2) Test Results

### a) Evaluation of Lung Functions

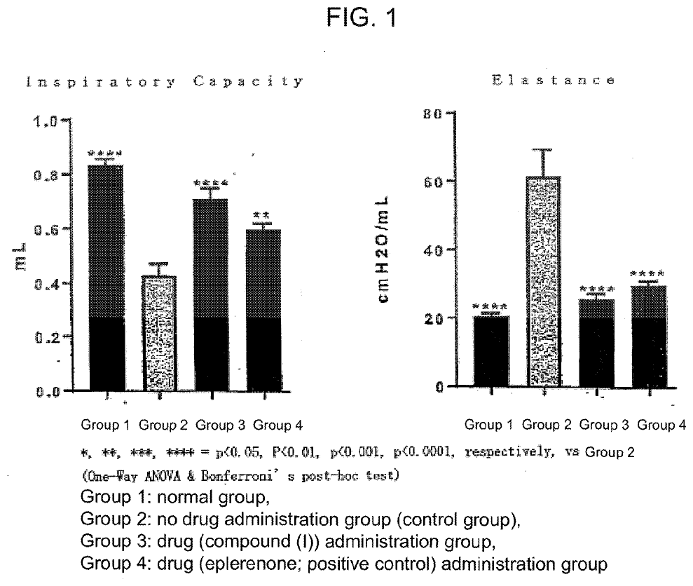

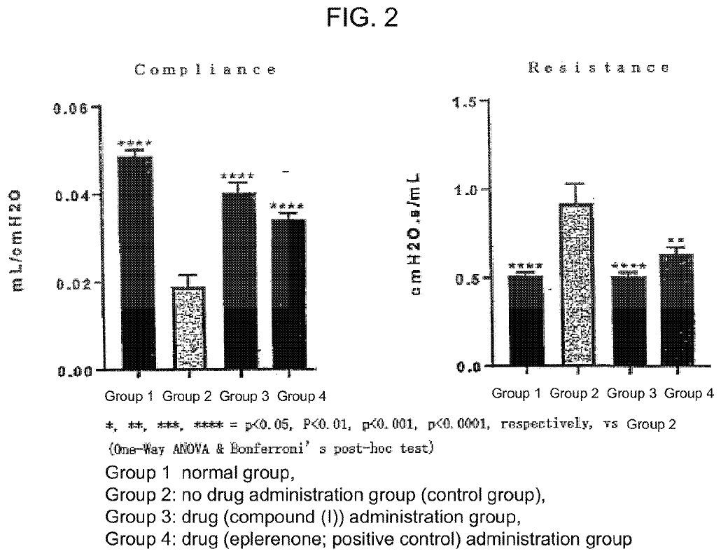

The test results are shown in FIGS. 1 and 2.

As shown in FIG. 1, bleomycin reduced the inspiratory capacity and increased the elastance (Group 2). Eleplarenone significantly improved such decreases in the lung functions due to bleomycin (Group 4).

In contrast, the compound (I) also showed an effect of significantly improving all lung functions and it was found that the inspiratory capacity in particular was significantly improved to an extent exceeding the effect of eplerenone (Group 3).

As shown in FIG. 2, bleomycin decreased the lung compliance and increased the resistance (Group 2). Eleplarenone significantly improved such decreases in the lung functions due to bleomycin (Group 4).

In contrast, the compound (I) also showed an effect of significantly improving all lung functions and it was found that the resistance in particular was very significantly improved to a level of the normal group exceeding the effect of eplerenone (Group 3).

From the above, in the present test, it was confirmed that, from a point of view of the lung functions, the compound (I) has an excellent improvement effect with respect to pulmonary fibrosis even as compared to eplerenone.

### b) Evaluation of Lung Pathological Tissue

### (i) Evaluation by Picrosirius Red Staining (Left Lung Lobes) and (ii) Evaluation of Hydroxyproline Content (μg) (Lower Lung Lobes and Middle Lung Lobe)

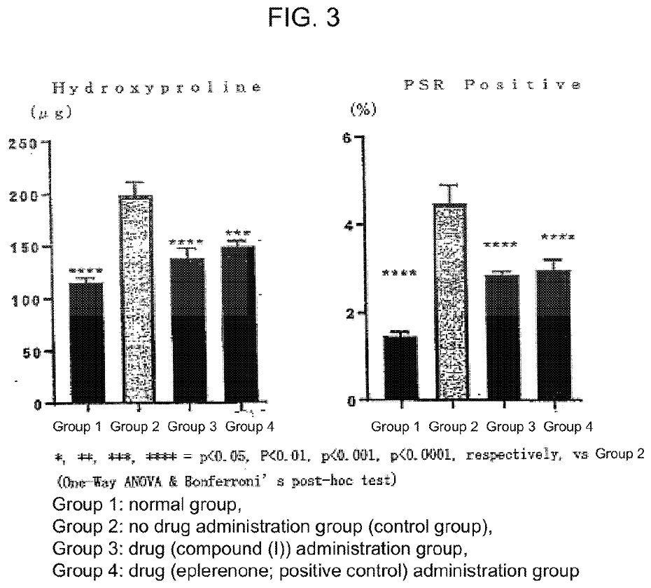

The test results are shown in FIG. 3.

As shown by an increase in a hydroxyproline content (hydroxyproline; μg) in the lower lung lobes and the middle lung lobe and an increase in a positive rate (PSR Positive %) in picrosirius red staining in the left lung lobes, bleomycin increased collagen accumulation in the lungs (Group 2). Such fibrosis by bleomycin was significantly improved by eplerenone (Group 4).

In contrast, the compound (I) also showed an effect of significantly improving pulmonary fibrosis in all evaluation items and it was found that the hydroxyproline content of the lung tissue in particular was significantly improved to an extent exceeding the effect of eplerenone (Group 3).

From the above, in the present test, it was confirmed that, even from a histopathological point of view, the compound (I) has an excellent improvement effect with respect to pulmonary fibrosis even as compared to eplerenone.

(iii) Evaluation of Gene Expression Fluctuation Related to Pulmonary Fibrosis

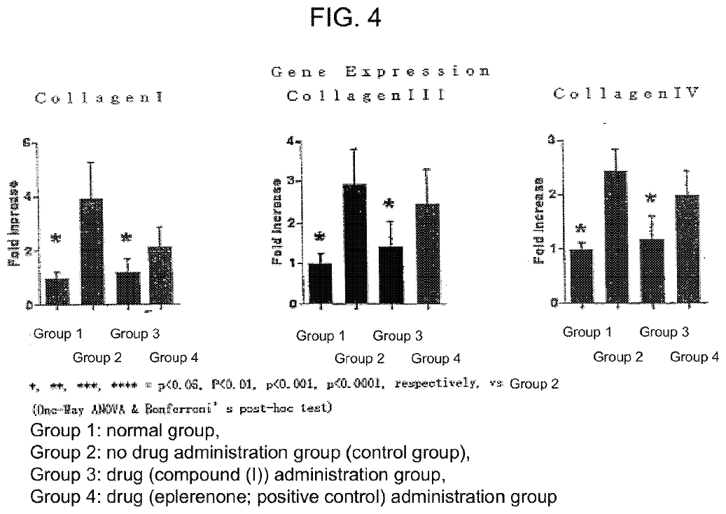

The test results are shown in FIGS. 4-7. By evaluating gene expression fluctuation related to pulmonary fibrosis, a therapeutic effect and a prophylactic effect of the compound (I) can be predicted.

Bleomycin increased gene expressions of type I collagen, type III collagen, and type IV collagen. Eplerenone could not reduce these gene expressions.

In contrast, the compound (I) significantly reduced these gene expressions (FIG. 4). From the above, in the present test, it was confirmed that, even from a point of view of controlling collagen production directly involved in pulmonary fibrosis, the compound (I) has an excellent improvement effect with respect to pulmonary fibrosis even as compared to eplerenone.

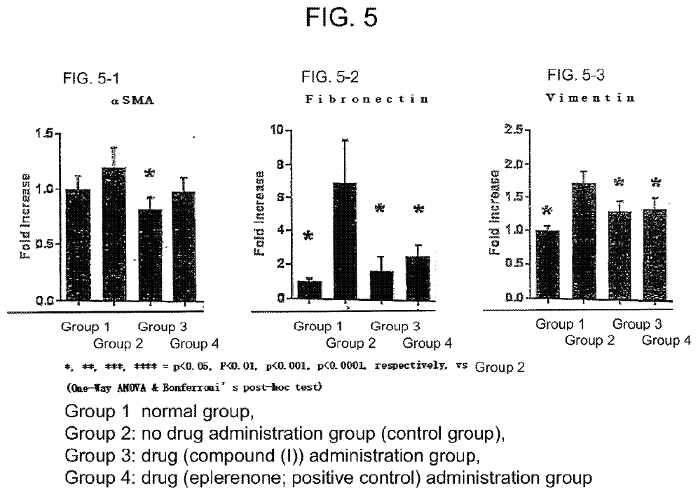

Bleomycin slightly increased αSMA gene expression. Eplerenone could not reduce this gene expression. In contrast, the compound (I) significantly reduced this gene expression (FIG. 5-1).

Bleomycin significantly increased fibronectin gene and vimentin gene expressions. Eplerenone significantly reduced these gene expressions as compared to bleomycin.

In contrast, the compound (I) significantly reduced these gene expressions (FIGS. 5-2 and 5-3).

From the above, in the present test, it was confirmed that the compound (I) has excellent control capability with respect to gene expressions of αSMA, fibronectin and vimentin involved in pulmonary fibrosis.

Bleomycin significantly increased gene expressions of CTGF, PAI-1 and Ccl2. Eplerenone significantly reduced these gene expressions as compared to bleomycin.

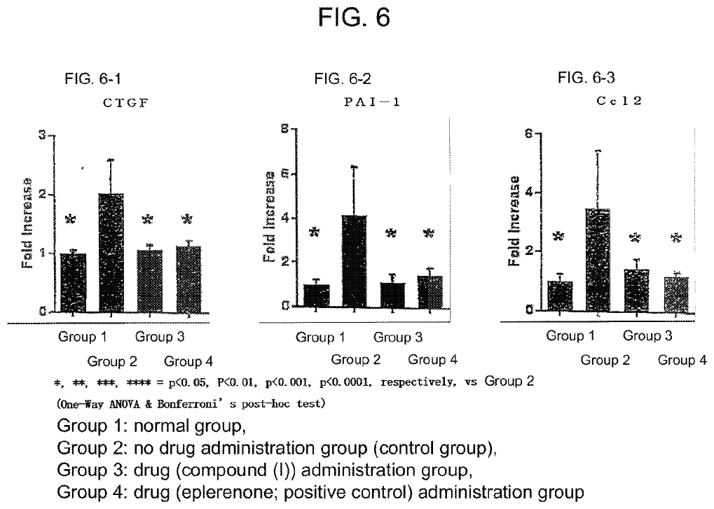

In contrast, the compound (I) significantly reduced these gene expressions (FIGS. 6-1 -6-3).

From the above, in the present test, it was confirmed that the compound (I) has excellent control capability with respect to gene expressions of CTGF, PAI-1 and Ccl2 involved in pulmonary fibrosis.

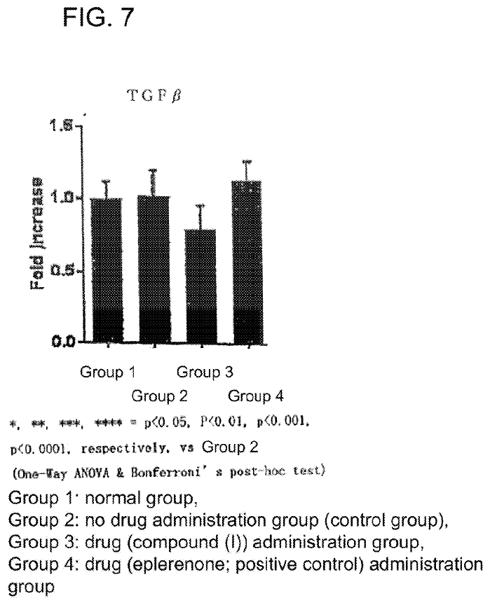

Bleomycin had no effect on gene expression of TGFβ. Eplerenone showed a tendency to increase the gene expression, whereas the compound (I) showed a tendency to decrease the gene expression (FIG. 7).

### c) Evaluation of Blood Samples

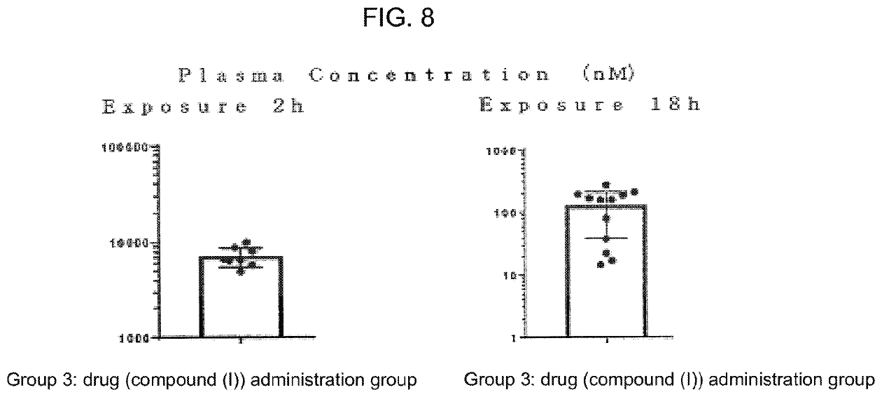

The test results are shown in FIG. 8.

At 2 hours and 18 hours after the final administration, a sufficient concentration of the compound (I) in a blood plasma was detected.

Such a finding of blood kinetics indicates that sufficient exposure in blood is obtained after the administration of the compound (I), and indicates usefulness of the compound (I) as a pulmonary fibrosis drug.

These results indicate that the compound (I) has a prophylactic or therapeutic effect for pulmonary fibrosis equal to or higher than that of eplerenone.

### Example 2: Antifibrotic Action in Bleomycin Subcutaneous Continuous Infusion Induced Mouse Pulmonary Fibrosis Model (2)

### (1) Test Method

On a day (Day 0) when the test was started, under anesthesia, an ALZET 1007D pump (storing therein bleomycin: 100 U/kg, or saline: 100 μl) (manufactured by ALZET) was embedded in an abdominal cavity of each C57/BL6NTAC mice (male, body weight 25-30 g at the start of the test) (manufactured by Taconic Biosciences Ltd.), and administration of bleomycin to a test group (48 mice) was started, and, on the other hand, administration of saline to a normal group (24 mice) (Group 1) was started, and, thereafter, the administrations were continued for 7 days (flow rate: 0.5 μl/hour) (the pump was removed on a tenth day (Day 10) after the start of the test). Body weights of the mice of the test group were measured daily, and, on the seventh day (Day 7) after the start of the test, the mice of the test group were randomly grouped as follows using an amount of decrease in body weight as an indicator.

Group 2: no drug administration group (control group) (12 mice)
Group 3: drug (the compound (I), 3 mg/kg) administration group (12 mice)
Group 4: drug (the compound (I), 10 mg/kg) administration group (12 mice)
Group 5: drug (the compound (I), 30 mg/kg) administration group (12 mice)

From an eighth day (Day 8) after the start of the test, oral gavage administration of a vehicle (0.1 ml per 10 g body weight) to the mice of Group 1 as the normal group and the mice of Group 2 as the no drug administration group was started, and, on the other hand, oral gavage administration of the compound (I) of a predetermined dosage to the mice of the drug administration groups of Groups 3-5 was started (for all groups, once a day in the morning). Thereafter, until a 21st day (Day 21) after the start of the test, the administrations were continued. After the administrations were completed, the following items were evaluated.

### a) Evaluation of Lung Function

Evaluation of 4 items including: 1) inspiratory capacity (mL); 2) compliance (mL/cmH₂O); 3) elastance) (cmH₂O/mL); and 4) resistance (cmH₂O.s/mL)

### b) Evaluation of Lung Pathological Tissue

### i) Evaluation by Picrosirius Red Staining (Left Lung Lobes)

### ii) Evaluation of Hydroxyproline Content (μg) (Right Lung Lobes)

### c) Evaluation of Blood Samples

Evaluation of a concentration (nM) of the compound (I) in a blood plasma at 2 hours (before the above evaluations) and at 24 hours after the final administration (Day 21) (similar to Example 1, blood samples obtained by tail vain blood sampling were evaluated.)

### (2) Test Results

### a) Evaluation of Lung Functions

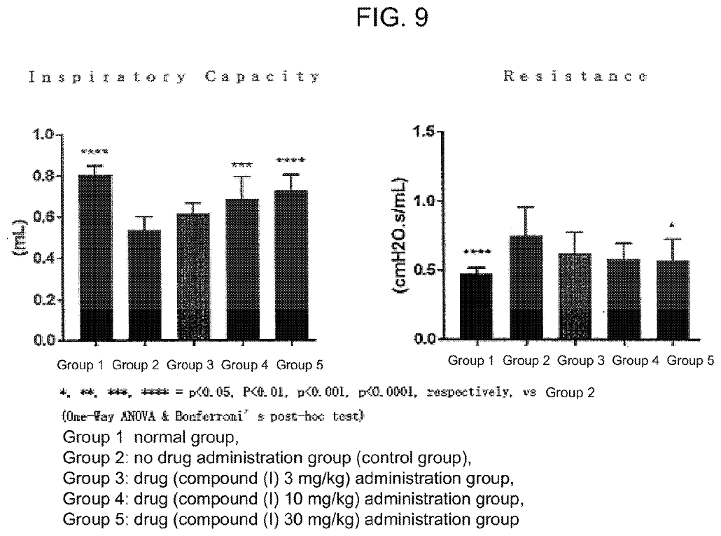

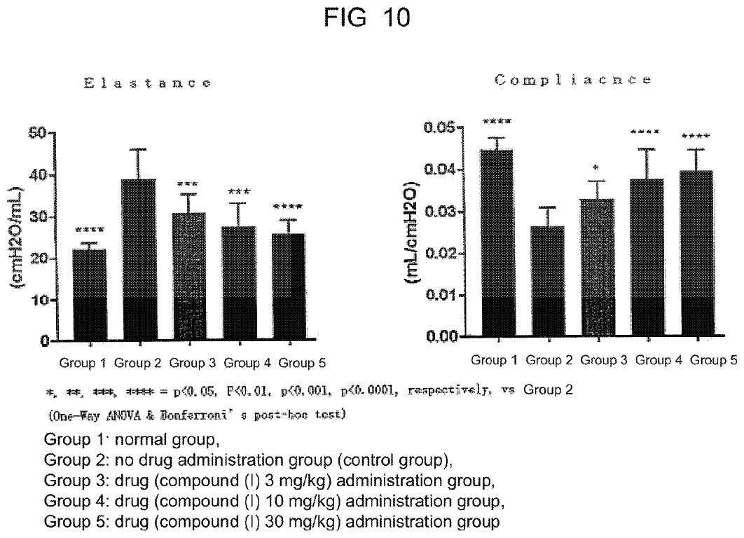

The test results are shown in FIGS. 9 and 10.

As shown in FIG. 9, bleomycin reduced the inspiratory capacity and increased the resistance (Group 2). In contrast, the compound (I) at doses of 10 mg/kg and 30 mg/kg significantly improved the inspiratory capacity (Groups 4 and 5), and the compound (I) at a dose of 30 mg/kg significantly improved the resistance (Group 5).

As shown in FIG. 10, bleomycin increased the elastance and decreased the compliance (Group 2). The compound (I) significantly improved such decreases of bleomycin in the lung functions of the two items in a dose-dependent manner (Groups 3-5).

From the above, in the present test, it was confirmed that, from a point of view of the lung functions, the compound (I) has an excellent improvement effect with respect to pulmonary fibrosis in a dose-dependent manner.

### b) Evaluation of Lung Pathological Tissue

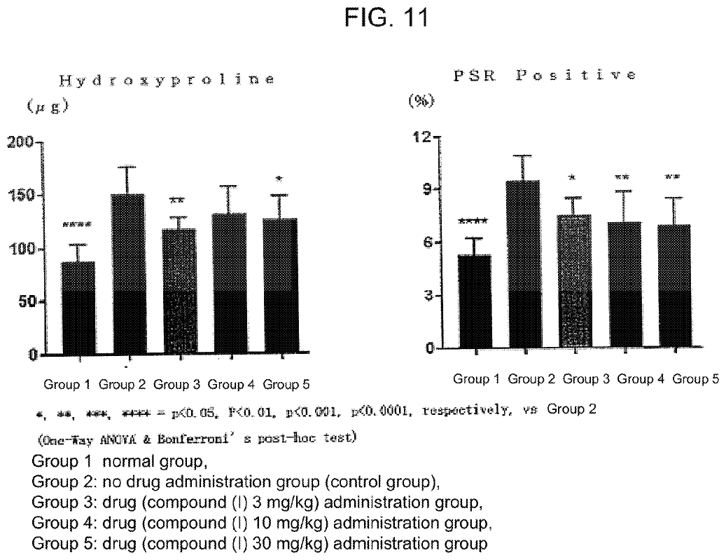

The test results are shown in FIG. 11.

As shown by an increase in a hydroxyproline content in the right lung lobes and an increase in a positive rate in picrosirius red staining in the left lung lobes, bleomycin increased collagen accumulation in the lung (Group 2). In contrast, the compound (I) at doses of 3 mg/kg and 30 mg/kg significantly decreased the hydroxyproline content (Groups 3 and 5), and the compound (I) at all the doses significantly decreased the positive rate in the picrosirius red staining (Groups 3-5).

From the above, in the present test, it was confirmed that, even from a histopathological point of view, the compound (I) has an excellent improvement effect with respect to pulmonary fibrosis in a dose-dependent manner.

### c) Evaluation of Blood Samples

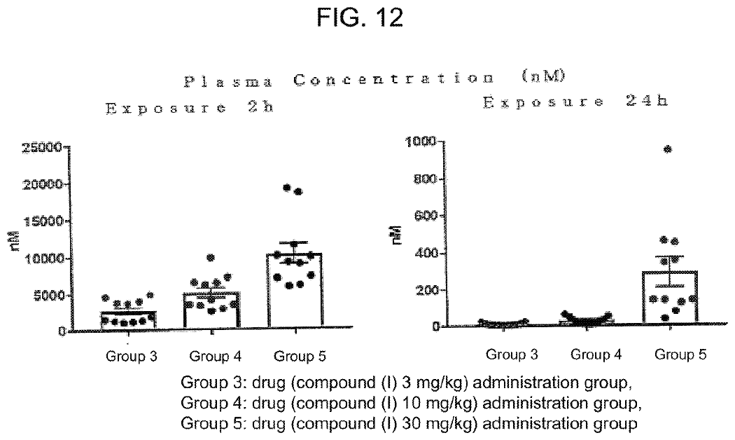

The test results are shown in FIG. 12.

At 2 hours after the final administration, the compound (I) showed a dose-dependent concentration in a blood plasma. At 24 hours after the final administration, for the compound (I) at a dose of 30 mg/kg, a sufficient concentration in a blood plasma was detected.

Such a finding of blood kinetics indicates that exposure of the compound (I) in blood depending on a dose is obtained, and indicates usefulness of the compound (I) as a pulmonary fibrosis drug.

These results indicate that the compound (I) has a dose-dependent effect, and indicate usefulness of the compound (I) as a pulmonary fibrosis drug.

Pulmonary fibrosis is mainly advanced fibroses of the lungs, and is a pulmonary disease that causes restrictive ventilatory impairment. Pulmonary fibrosis is thought to be caused by that, due to that inflammation in lung interstitium is repeated, an abnormal damage repair reaction with respect to continuous alveolar epithelial cell damage is repeated.

There may be cases where a cause for inflammation in the lung interstitium such as infection, a collagen disease, radiation, drugs, dust or the like can be identified, and there may be cases where the cause cannot be identified. However, when an inflamed tissue has become fibrotic, it progresses to pulmonary fibrosis. Here, pulmonary fibrosis for which a cause cannot be identified is referred to as idiopathic pulmonary fibrosis. Idiopathic pulmonary fibrosis is also frequent and has poor prognosis, and most often develops in adults 50 years of age or older, and causes irreversible fibrosis in the lungs and is fatal by reducing the respiratory function.

For treatment of pulmonary fibrosis, in general, steroid drugs and immunosuppressants have been used. However, findings have been accumulated that, in particular, for idiopathic pulmonary fibrosis, when steroid drugs or immunosuppressants are used over a long period of time, fibrosis is rather worsened. For pulmonary fibrosis for which there are various causes or a cause is unknown, there is no drug treatment that can be widely recommended for treatment of pulmonary fibrosis in general.

In recent years, as new types of idiopathic pulmonary fibrosis therapeutic medicaments, antifibrotic agents (for example, pirfenidone, nintedanib) are on the market. However, there is a problem that both drugs have strong side effects. For example, with respect to pirfenidone, it is necessary to use the drug taking into account of a possibility of carcinogenesis of skin due to exposure to light, and treatment options are still limited in actuality for this severe disease. On the other hand, it has been reported that spironolactone which is a steroid type mineralocorticoid receptor antagonist (MR antagonist) improves accumulation of hydroxyproline which is an indicator of tissue fibrosis in bleomycin-induced pulmonary fibrosis mice (see European Journal of Pharmacology, 718 (2013), 290-298; PLOS ONE, 8 (11) (2013), e81090; and Nanomedicine (Lond.), 11 (11) (2016), 1393-1406). However, there is a concern about side effects such as gynecomastia due to steroid drugs, and clinical usefulness for pulmonary fibrosis (particularly idiopathic pulmonary fibrosis) in human is unknown.

As described above, an effective treatment for pulmonary fibrosis has not been sufficiently established at present and early establishment of a treatment by developing new drugs having high safety and high therapeutic effectiveness is strongly desired.

In International Publication No. 2007/089034, a 1,4-benzoxazine compound containing the compound (I) of the present invention is described, and it is described that the compound (I) is used as an MR antagonist. Further, in International Publication No. 2018/062134, it is described that the compound (I) is used for treatment of a non-alcoholic fatty liver disease (NAFLD), non-alcoholic steatohepatitis (NASH), and the like. However, none of the literatures has specifically mentioned the application of the compound (I) of the present invention to pulmonary fibrosis.

As described above, a drug treatment for pulmonary fibrosis has not yet been well established, and development of a novel prophylactic or therapeutic medicament has been an urgent task in the technical field of medicine.

According to the present invention, a medicament that contains the compound (I) as an active ingredient and that can be effectively and safely used for prophylaxis or treatment of pulmonary fibrosis can be provided.

Unless otherwise noted, technical terms and scientific terms used in the present specification have the same meaning as commonly understood by a person skilled in the art to which the present invention belongs.

The present invention provides a novel medicament that contains the compound (I) as an active ingredient and that can be effectively and safely used for prophylaxis or treatment of pulmonary fibrosis.

Obviously, numerous modifications and variations of the present invention are possible in light of the above teachings. It is therefore to be understood that within the scope of the appended claims, the invention may be practiced otherwise than as specifically described herein.

What is claimed is:

1. A medicament for prophylaxis or treatment of pulmonary fibrosis, comprising:
a compound of formula (I) 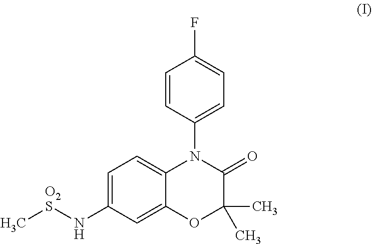
or a pharmaceutically acceptable salt thereof.

2. The medicament according to claim 1, wherein the pulmonary fibrosis is an interstitial lung disease with fibrosis.

3. The medicament according to claim 2, wherein the interstitial lung disease with fibrosis is a disease selected from the group consisting of idiopathic pulmonary fibrosis, disease induced lung fibrosis and other factor induced lung fibrosis.

4. A method for prophylaxis or treatment of pulmonary fibrosis, comprising:
administering a prophylactically or therapeutically effective amount of a compound of formula (I) 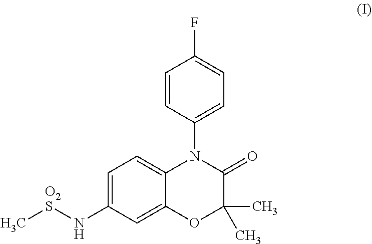
or a pharmaceutically acceptable salt thereof to a patient in need thereof.

5. A compound of formula (I) 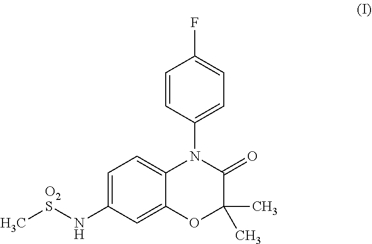
or a pharmaceutically acceptable salt thereof for prophylaxis or treatment of pulmonary fibrosis.

6. Use of a compound of formula (I) 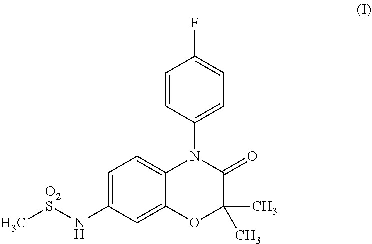
or a pharmaceutically acceptable salt thereof for manufacturing a medicament for prophylaxis or treatment of pulmonary fibrosis.
